(19) United States
(12) Patent Application Publication
(10) Pub. No.: US 2022/0192559 A1
(43) Pub. Date: Jun. 23, 2022

(54) ELECTRONIC DEVICE, METHOD OF DETERMINING MENTAL STATE OF USER IN CONSIDERATION OF EXTERNAL MENTAL LEVEL ACCORDING TO INPUT BEHAVIOR OF USER, AND COMPUTER PROGRAM

(71) Applicant: RFCAMP LTD., Gyonggi-do (KR)

(72) Inventors: Jae Hyung RYU, Gyeonggi-do (KR); Kwon Soo KIM, Seoul (KR); Ji Youn KIM, Gyeonggi-do (KR)

(73) Assignee: RFCAMP LTD., Gyonggi-do (KR)

(21) Appl. No.: 17/539,055

(22) Filed: Nov. 30, 2021

(30) Foreign Application Priority Data
Dec. 18, 2020 (KR) 10-2020-0178917

(51) Int. Cl. A61B 5/16; A61B 5/00; G16H 10/20; G16H 20/70

(52) U.S. Cl. CPC A61B 5/165; G16H 20/70; G16H 10/20; A61B 5/7435

(57) ABSTRACT

Provided is a method of determining a mental state of a user in consideration of an external mental level according to an input behavior of the user.

### CROSS-REFERENCE TO RELATED APPLICATION

This application is based on and claims priority under 35 U.S.C. § 119 to Korean Patent Application No. 10-2020-0178917, filed on Dec. 18, 2020, in the Korean Intellectual Property Office, the disclosure of which is incorporated by reference herein in its entirety.

### BACKGROUND

### 1. Field

Embodiments of the present disclosure relate to an electronic device, a method of determining a mental state of a user in consideration of an external mental level according to an input behavior of the user, and a computer program.

### 2. Description of the Related Art

Due to the development of mobile technology and the spread of the global epidemic of Covid-19, society is entering a phase in which face-to-face human relationships that have been a main approach are decreasing and non-face-to-face human relationships are rapidly becoming common. This rapid change in interpersonal relationships, which is unprecedented in human history, is producing many people who cannot cope with the speed of the change and is causing serious social problems such as depression and suicide.

In the case of South Korea, which has maintained the world's highest suicide rate, the suicide rate among young adults and middle-aged people has been increasing recently. Thus, it may be a natural trend that the technological effort to identify the mentality of a user in mobile applications on smartphones, which have become an indispensable necessity, rather than food, clothing, and shelter. Accordingly, various business attempts are being made to find mental patterns of the user in application usage and selection patterns of the user in applications.

A mental state of an application user, which is determined through one more user inputs in an application and one more behaviors of the user in an application, may be identified more objectively and precisely by analyzing the relationship patterns between an external energy level (external mental level) that may be extracted from an input itself and an internal energy level (internal mental level) that is latent behind the input behavior.

### SUMMARY

Embodiments of the present disclosure provide an electronic device, a method of determining a mental state of a user in consideration of an external mental level according to an input behavior of the user, and a computer program.

Additional aspects will be set forth in part in the description which follows and, in part, will be apparent from the description, or may be learned by practice of the presented embodiments of the disclosure.

According to embodiments of the present disclosure, a method may include: generating, by an electronic device, output data providing a list of a plurality of pieces of emotion content and displaying the output data; receiving, by the electronic device, a selection input for selecting a predetermined quantity of pieces of emotion content from among the plurality of pieces of emotion content in the output data; calculating, by the electronic device, an internal mental level based on positive level values and stimulation level values of the selected predetermined quantity of pieces of emotion content; calculating, by the electronic device, an external mental level based on a movement of the selection input for selecting the predetermined quantity of pieces of emotion content; and determining, by the electronic device, a mental state of the user in consideration of the internal mental level and the external mental level.

The output data may include a user interface in which a plurality of pieces of emotion content are arranged in a plurality of rows and a plurality of columns in a predetermined arrangement, and the arrangement of the plurality of pieces of emotion content may be determined by an administrator and may be fixed.

The calculating of the external mental level may include calculating, based on coordinate values of the selected predetermined quantity of pieces of emotion content, a physical distance value between the predetermined quantity of pieces of emotion content and a movement speed value, and calculating the external mental level in consideration of the physical distance value between the predetermined quantity of pieces of emotion content or the movement speed value.

According to embodiments of the present disclosure, the method may further include calculating, by the electronic device, a degree of relevance between internal and external mental states in consideration of the internal mental level and the external mental level, and calculating a reliability of the internal mental level of the user based on the degree of relevance between the internal and external mental states.

According to embodiments of the present disclosure, the method may further include changing, by the electronic device, the mental state of the user based on a degree of relevance between internal and external mental states for each user.

According to embodiments of the present disclosure, the method may further include calculating, by the electronic device, a degree of relevance between internal and external mental states based on the internal mental level and the external mental level, when the degree of relevance between the internal and external mental states exceeds a preset first reference value, setting, to ‘high’, the reliability of the internal mental level based on the predetermined quantity of pieces of emotion content selected by the user, and when the degree of relevance between the internal and external mental states is less than or equal to a second reference value, setting, to ‘low’, the reliability of the internal mental level based on the predetermined quantity of pieces of emotion content selected by the user.

The first and second reference values may be set for each user or set based on at least one of age, gender, occupation, and residential area of the user.

According to embodiments of the present disclosure, from among the predetermined quantity of pieces of emotion content, pieces of emotion content provided at once may include pieces of emotion content classified into a predetermined positive level value and stimulation level value.

According to embodiments of the present disclosure, an electronic device may include: a provision data generator configured to generate output data providing a list of a plurality of pieces of emotion content and display the output data; an emotion path inputter configured to receive a selection input for selecting a predetermined quantity of pieces of emotion content from among the plurality of pieces of emotion content in the output data; an internal mental level determiner configured to calculate an internal mental level based on positive level values and stimulation level values of the selected predetermined quantity of pieces of emotion content; an external mental level determiner configured to calculate an external mental level based on a movement of the selection input for selecting the predetermined quantity of pieces of emotion content; and a data processor configured to determine a mental state of a user in consideration of the internal mental level and the external mental level.

According to an embodiment of the present disclosure, a computer program may be stored in a medium to execute, by using a computer, any one of the methods of determining a mental state of a user in consideration of an external mental level according to an input behavior of the user, according to an embodiment of the present disclosure.

In addition, another method and another system implementing the present disclosure, and a computer-readable recording medium having recorded thereon a computer program for executing the method are further provided.

Other aspects, features and advantages other than those described above will become apparent from the following drawings, claims and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of a mentality determiner according to embodiments of the present disclosure;

FIG. 2 is a diagram of a screen providing pieces of provided emotion content, according to embodiments of the present disclosure;

FIG. 3 is a diagram of an example of a graph illustrating a relationship between stimulation level values and positive level values for pieces of emotion content;

FIG. 4 is a diagram of an example of emotion content selected to be provided according to a certain criterion;

FIG. 5 is a diagram illustrating a selection input moving between emotion content in a first column and emotion content in a fifth column;

FIG. 6 is a diagram illustrating pieces of selected emotion content and internal mental level values calculated by the pieces of emotion content;

FIG. 7 is a table representing coordinate values, internal coordinate values, external difference values, internal difference values, input time values, external speed values, and internal speed values, according to embodiments of the present disclosure;

FIG. 8 is a block diagram of an electronic device including a mentality determiner;

FIG. 9 is a diagram of a network environment of a mentality determination system;

FIG. 10 is a flowchart of a mentality determination method according to embodiments of the present disclosure; and

FIG. 11 is an exemplary diagram of statistical data for ‘in a short time’ and ‘Moebius strip’, which are exemplary emotional content.

FIG. 12 is an exemplary diagram of a change trend in positivity and sensitivity of a user.

FIG. 13 is an exemplary diagram of a user's emotional fluctuations.

FIG. 14 is an exemplary diagram of a change trend of emotional contents selected by a user.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the configuration and operation of the present disclosure will be described in detail with reference to embodiments of the present disclosure illustrated in the accompanying drawings.

As the present disclosure allows for various changes and numerous embodiments, particular embodiments will be illustrated in the drawings and described in detail in the written description. The effects and features of the present disclosure and the accomplishing methods thereof will become apparent from the embodiments described below in detail with reference to the accompanying drawings. The present disclosure may, however, be embodied in many different forms and should not be construed as being limited to the embodiments set forth herein.

The embodiments will be described below in more detail with reference to the accompanying drawings. Those components that are the same or are in correspondence are rendered the same reference numeral regardless of the figure number, and redundant explanations are omitted.

It will be understood that the terms used herein, such as “training,” “learning,” etc. are not intended to refer to mental actions such as human educational activities but refer to performing machine learning through computing according to procedures.

Also, it will be understood that although the terms “first,” “second,” etc. may be used herein to describe various components, these components should not be limited by these terms. These components are only used to distinguish one component from another.

As used herein, the singular forms “a,” “an,” and “the” are intended to include the plural forms as well, unless the context clearly indicates otherwise.

In the following embodiments, it is to be understood that the terms such as “including” and “having” are intended to indicate the existence of the features, or elements disclosed in the present disclosure, and are not intended to preclude the possibility that one or more other features or elements may exist or may be added.

Also, sizes of elements in the drawings may be exaggerated or reduced for convenience of explanation. In other words, since sizes and thicknesses of components in the drawings are arbitrarily illustrated for convenience of explanation, the following embodiments are not limited thereto.

When a certain embodiment may be implemented differently, a particular process order may be performed differently from the described order. For example, two consecutively described processes may be performed substantially at the same time or may be performed in an order opposite to the described order.

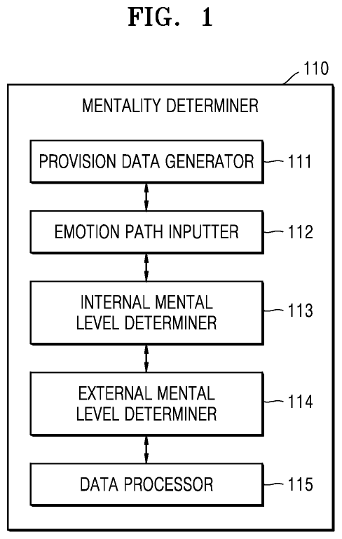

FIG. 1 is a block diagram of a mentality determiner 110 according to embodiments of the present disclosure.

The mentality determiner 110 may include a provision data generator 111, an emotion path inputter 112, an internal mental level determiner 113, an external mental level determiner 114, and a data processor 115.

The mentality determiner 110 may provide one or more pieces of emotion content representing mental states, and determine a mental state of a user based on pieces of emotion content selected by the user from among the one or more pieces of emotion content. In this case, the emotion content may have various formats, such as text, images, icons, moving icons, videos, auditory data, etc. The emotion content may include emotion words, emotion images, emotion icons, emotion sounds, etc.

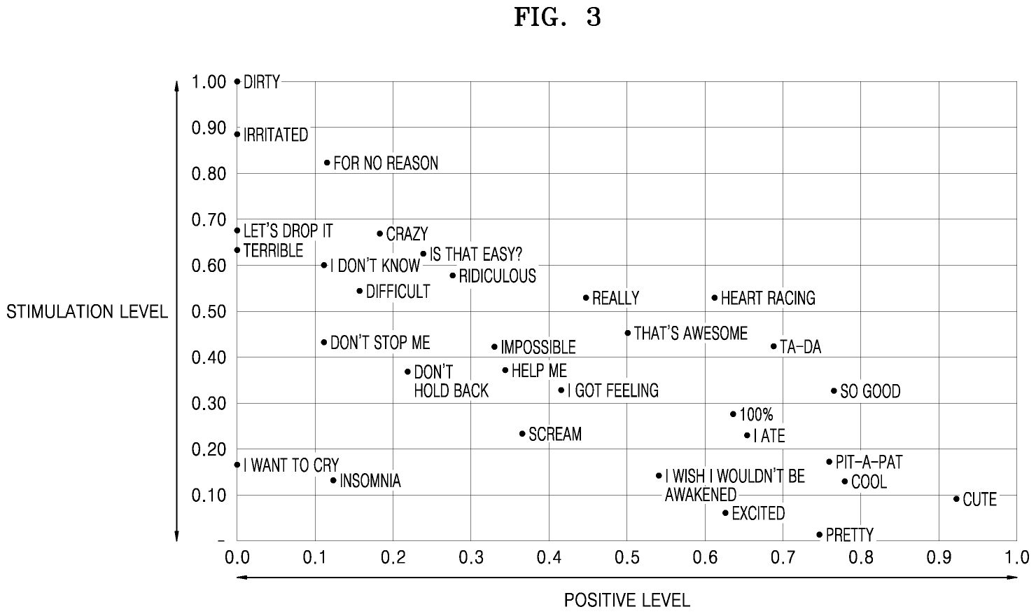

The emotion content may be stored as stimulation level values or positive level values. As illustrated in FIG. 3, examples of the emotion content may include words including emotions, such as “dirty”, “irritated”, “it's been so long”, “coochy coo”, “I miss you”, “somehow”, “lethargic”, “obsession”, “help me”, “this is a fact”, “exhausted” and the like. For each emotional content, a graph and a table of stimulation level values and positive level values may be stored. The stimulation level values and the positive level values may be called internal coordinate values of the corresponding emotion content. A graph and a table representing stimulation levels and positive levels corresponding to the emotion content may be stored in an external server or database and managed. FIG. 3 illustrates a graph and a table of stimulation levels and positive levels of the emotion content in the form of words, but is not limited thereto. There may also be a graph and a table of stimulation levels and positive levels for various types of content such as images, emoticons, and icons. The stimulation levels and the positive levels may be set as numerical values or may be set as strong, medium, and low.

The provision data generator 111 may generate output data provided, in response to a login input from a user and/or an emotion input signal from a user.

The provision data generator 111 may select one or more pieces of emotion content to be provided and generate output data in which the one or more pieces of emotion content are composed in a predetermined layout.

The provision data generator 111 may receive one or more pieces of emotion content to be provided from a remote server or database, and generate output data by using the received pieces of emotion content.

The provision data generator 111 may receive data on a form of providing emotion content from the remote server or database, and generate output data of one or more pieces of emotion content based on the received data.

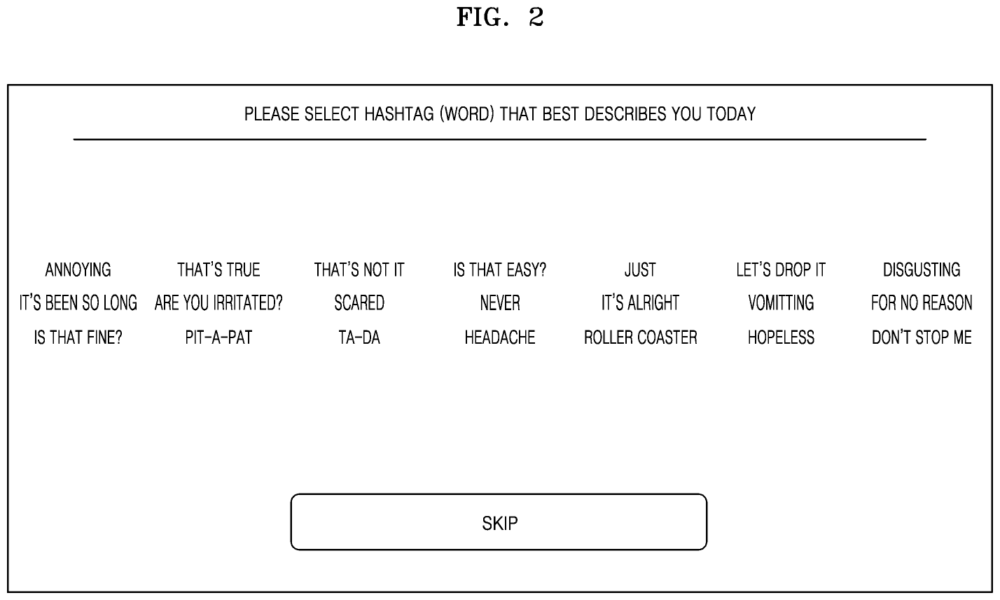

As illustrated in FIG. 2, output data for selecting today's emotion content may be provided in response to a login input of the user.

In another embodiment, the one or more pieces of emotion content to be provided may be selected by a rule or a regulation stored in advance in the mentality determiner 110 or may be selected in response to a signal from a terminal of a user with management authority. The form of providing pieces of emotion content may also be selected by the rule or the regulation stored in advance in the mentality determiner 110 or may be selected in response to a signal from the terminal of the user with management authority.

The rule or the regulation applied to selection of the one or more pieces of emotion content to be provided may designate a group of pieces of emotion content to be provided or may be designated based on attributes of the pieces of emotion content to be provided. For example, the pieces of emotion content to be provided may be designated as having a first stimulation level and a first positive level, or having a stimulation level of a first group and a positive level of the first group, and the like. Each emotion content may be stored in relation to a corresponding positive level value and stimulation level value.

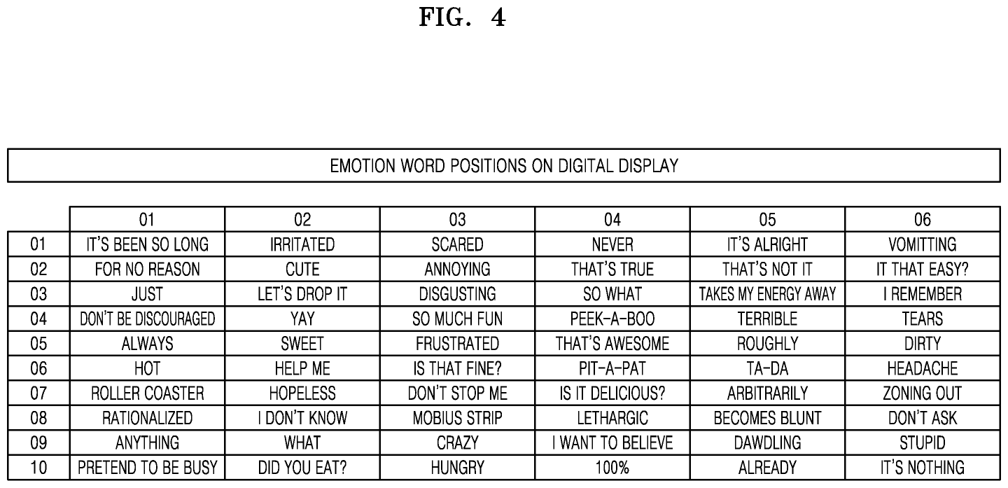

As illustrated in FIG. 4, the pieces of emotion content may be provided in the form of a matrix. In a matrix of a plurality of columns and rows, each emotion content may be displayed at a designated position. According to a predetermined format of presentation, a first row may be displayed in the following order: “it's been so long”, “irritated”, “scared”, “never”, “it's alright”, and “vomiting”.

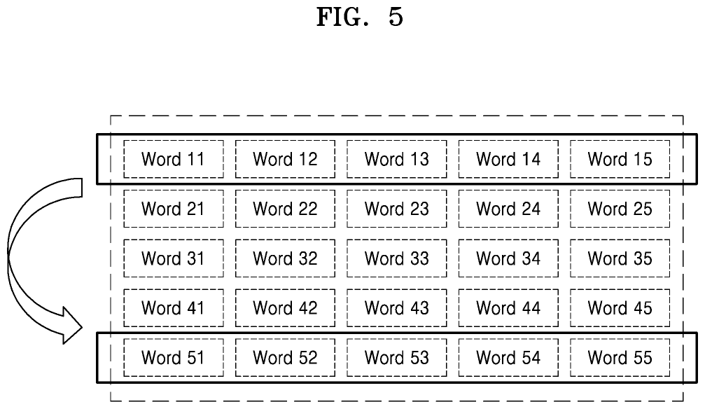

The pieces of emotion content is provided in the form of a matrix, but as in FIG. 5, the number of rows or columns displayed at a time may be limited. Since the emotional contents are displayed in a limited row or column, the user should input an input for moving the column or row of the matrix in order to select the emotional contents displayed in another column or another row. For example, when pieces of emotion content in the first row are displayed, in order to select pieces of emotion content in a fifth row, an input changing a displayed row from the first row to the fifth row is received from the user.

The emotional contents and/or the arrangement of the emotional contents displayed at once may be determined by a rule. The emotional contents displayed at once time may be contents having a stimulus level value of a predetermined protocol and/or a positive level value.

The emotion path inputter 112 may input a selection input for selecting emotion content from among output data. The emotion path inputter 112 may measure one more degree of movement corresponding to the selection inputs. The degree of movement of the selection inputs may be measured based on, when first to fifth emotion content is selected, the number of inputs that a row moves, the number of inputs that a column moves, etc. while the first to fifth emotion content is selected.

The emotion path inputter 112 may measure a selection time interval of the selection inputs. The selection time interval of the selection input may be a time interval from the time when the emotion content is initially selected to the time when the selection of the emotion content is finished.

The emotion path inputter 112 may obtain the selection input for selecting the emotion content and calculate the degree of movement, and the selection time interval.

The internal mental level determiner 113 may calculate the current internal mental level of the user based on the pieces of selected emotion content. The internal mental level may be calculated based on positive level values and stimulation level values of the pieces of selected emotion content.

The external mental level determiner 114 may determine an external mental level based on the selection input for selecting the pieces of emotion content. The external mental level may be determined based on at least one of the selection inputs, the degree of movement of the selection inputs, and the selection time interval of the selection inputs. In another embodiment, the external mental level determiner 114 may calculate a movement speed based on the degree of movement and the selection time interval, and may determine the external mental level based on the movement speed.

The data processor 115 may determine a mental state of each user based on the internal mental level and the external mental level.

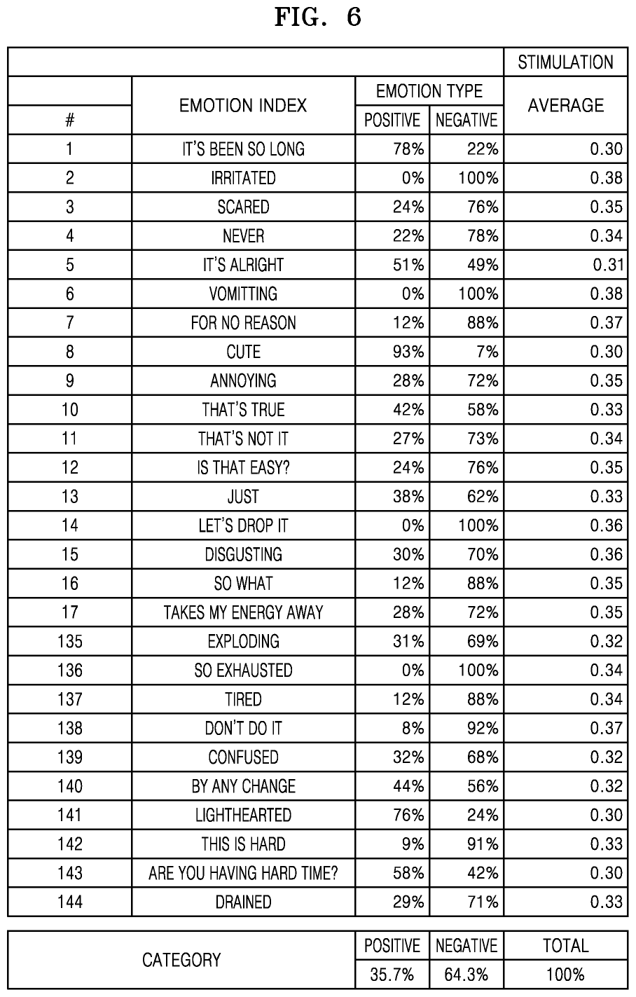

The data processor 115 may calculate, as illustrated in FIG. 6, the proportions of positives and negatives in a current mental state of the user, which is the internal mental level, based on the positive level values of the pieces of selected emotion content.

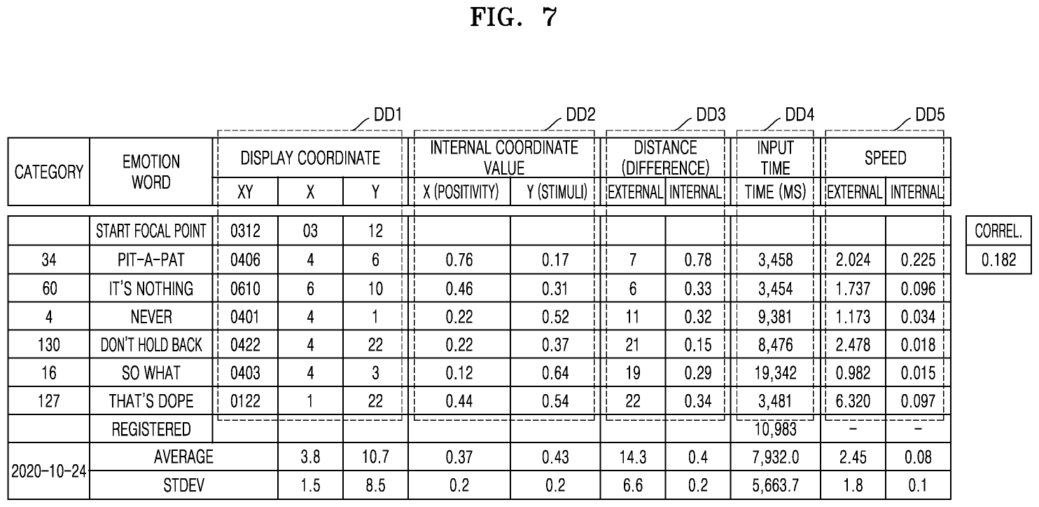

As illustrated in FIG. 7, when pieces of emotion content, such as a pit-a-tat 34, it's nothing 60, never 4, don't hold back 130, so what 16, and that's dope 127, are selected, the emotion path inputter 112 may extract a coordinate value of each emotion content, like DD1. The emotion path inputter 112 may extract positive level values and stimulation level values of the pieces of selected emotion content, like DD2. The emotion path inputter 112 may sequentially calculate distances and difference values between the pieces of selected emotion content as movement (physical) difference values based on movement related to the external mental level and difference values of emotion level values related to the internal mental level (DD3). The emotion path inputter 112 may extract values of time at which the pieces of emotion content are input, like DD4. The emotion path inputter 112 may calculate the speed of an external mental movement and the speed of an internal mental change, which are obtained by dividing the distance and difference values of the pieces of emotion content by the input time, like DD5.

The data processor 115 may calculate a degree of relevance between internal and external mental states between a value for the external mental level and a value for the internal mental level. The degree of relevance between the internal and external mental states may be managed by user, by date, by month, by season, by age, and by gender. The data processor 115 may more accurately determine the mental state of the user in further consideration of the degree of relevance between the internal and external mental states in addition to the external mental level and the internal mental level. The degree of relevance between the internal and external mental states may be set for each user based on a mental state selection process performed in the past for the corresponding user. For the corresponding user, when there is no history of mental state selection made in the past, the degree of relevance between the internal and external mental states corresponding to user-related information, such as age, gender, occupation, and residential area of the corresponding user, and external environment information (weather, season, temperature, humidity, illuminance, date, etc.) related to the time when the corresponding history of mental state selection is made may be further considered.

The data processor 115 may determine the reliability of a mental state selection input made by the user based on the degree of relevance between the internal mental level and the external mental level of the user. For example, when the degree of relevance between the internal mental level and the external mental level is lower than a preset reference value, the reliability of the internal mental level based on the pieces of emotion content selected by the user may be set to ‘low’. When the degree of relevance between the internal mental level and the external mental level exceeds a preset reference value, the reliability of the internal mental level based on the pieces of emotion content selected by the user may be set to ‘high’. The degree of relevance may be individually determined based on the user-related information, such as age, gender, occupation, and residential area of the user, and the external environment information (weather, season, temperature, humidity, illuminance, and date, etc.) related to the time when the corresponding history of mental state selection is made, but is not limited thereto.

In another embodiment, the mentality determiner 110 may determine the mental state of the user based on a direct input (typing, and file attachment, etc.) of the emotion content as well as a selection input for selecting the pieces of emotion content.

In another embodiment, when an emotion coordinate value of input emotion content is not stored in advance, the mentality determiner 110 may set the emotion coordinate value of the corresponding emotion content based on a previously stored emotion coordinate value of another emotion content or may request, and set, the emotion coordinate value of the corresponding emotion content from an external device.

When a position coordinate value of the corresponding emotion content is changed, the mentality determiner 110 may also change and calculate a distance value between the pieces of emotion content, a movement degree value, and a speed value. The distance value between the pieces of emotion content and the movement degree value may be calculated based on a coordinate value at the time input by the user.

In another embodiment, the mentality determiner 110 may determine the mental state of the user based on an input from a user other than pieces of provided emotion content. The mentality determiner 110 may determine the mental state of the user based on pieces of attribute information such as a signature input and a drawing input from the user.

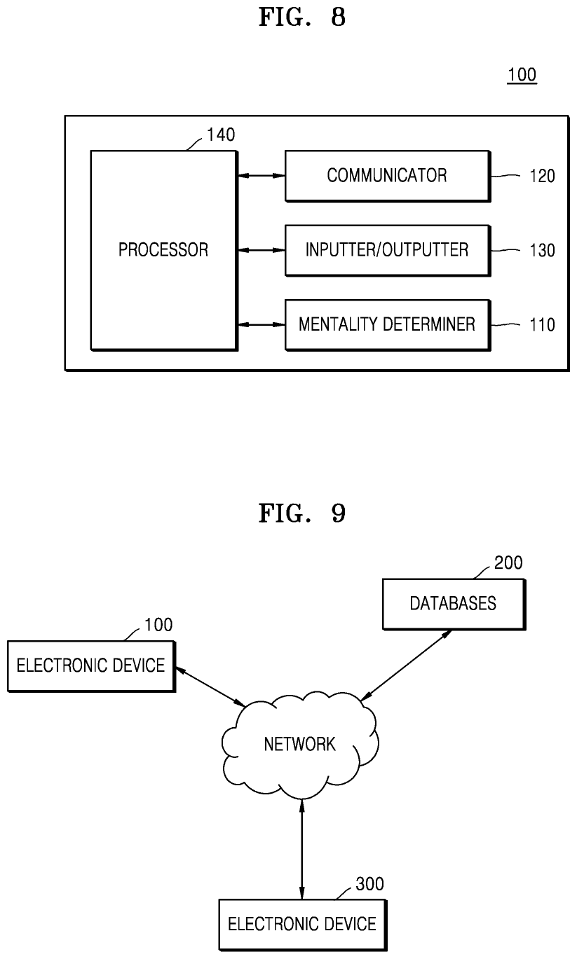

FIG. 8 is a block diagram of an electronic device 100 including a mentality determiner.

The electronic device 100 may include the mentality determiner 110, a communicator 120, an inputter/outputter 130, and a processor 140.

The mentality determiner 110 is implemented as illustrated in FIG. 1 and may be a set of one or more instructions. The mentality determiner 110 may be implemented as a computer-readable medium. The mentality determiner 110 may be a random access memory (RAM), a read only memory (ROM), and a permanent mass storage device such as a disk drive. The mentality determiner 110 may be a computer-readable recording medium such as a floppy drive, a disk, a tape, a digital video disc/compact disc read-only memory (DVD/CD-ROM) drive, or a memory card.

The communicator 120 may provide a function for communicating with an external device via a network. For example, a request generated by the processor 140 of the electronic device 100 according to a program code stored in a recording device such as the mentality determiner 110 may be transmitted to electronic devices, databases, or other user terminals via the network under the control by the communicator 120. For example, a control signal or instruction received through the communicator 120 may be transmitted to the processor 140, a storage medium, or the mentality determiner 110, and a received video image may be stored in a storage medium or the mentality determiner 110.

The inputter/outputter 130 may display a screen providing information or receive an input from the user. For example, the inputter/outputter 130 may include an operation panel for receiving a user input and a display panel for displaying a screen.

In detail, an inputter may include devices capable of receiving various types of user input, such as a keyboard, a physical button, a touch screen, a camera, or a microphone. Also, an outputter may include a display panel or a speaker. However, the present disclosure is not limited thereto, and the inputter/outputter 130 may include a configuration that supports various inputs/outputs.

The processor 140 may be implemented as one or more processors, and may be configured to process instructions of a computer program by performing basic arithmetic, logic, and input/output operations. The instructions may be provided to the processor 140 by the storage medium or the communicator 120. For example, the processor 140 may be configured to execute a received instruction according to a program code stored in the mentality determiner 110 or the recording device such as a storage medium.

The electronic device 100 may further include a computer-readable recording medium, such as a RAM and a ROM, and a permanent mass storage device such as a disk drive.

FIG. 9 is a diagram of a network environment of a mentality determination system.

The mentality determination system may include the electronic device 100 including the mentality determiner 110, a database 200, and an electronic device 300.

The electronic device 100 may be connected to another user terminal 300 and the database 200 through a network to transmit and receive data. The electronic device 100 including the mentality determiner 110 may calculate an internal mental level and an external mental level corresponding to a user input, which is input through an inputter/outputter, and determine a mental state of a user.

The electronic device 100 may receive a selection input from another electronic device 300 electrically connected thereto. The electronic device 100 may receive data required to determine the mental state of the user from the database 200.

The database 200 may store and manage pieces of emotion content to be provided. The database 200 may store stimulation level values and positive level values of the pieces of emotion content, and coordinate values of the pieces of emotion content.

The database 200 may manage rules and regulations related to selection of emotion content.

The database 200 may store a history of a selection input of emotion content selected by the user. The history of the selection input of the emotion content may include pieces of selected emotion content, a path of the selection input, a movement difference value, an internal mental difference value, a selection movement speed value, an internal mental change value, etc.

The database 200 may store and manage user-related information managed for each user. The database 200 may store and manage information related to external environment variables. The database 200 may store and manage a degree of relevance between internal and external mental states corresponding to the user-related information and the information related to the external environment variables.

The database 200 may manage a reference value for determining the reliability of the internal mental level for each user based on the user-related information and the information related to the external environment variables.

The database 200 may group users based on the mental state of the user, information related to internal and external mental states of the user, and the like.

The electronic device 300 may receive the internal mental level and the external mental level of the user from the electronic device 100, receive a mental state value of the user, and perform a function of managing mental states of users. The electronic device 300 may group one or more users based on today's mental state, an internal mental level, or an external mental level, and may create and manage a single community.

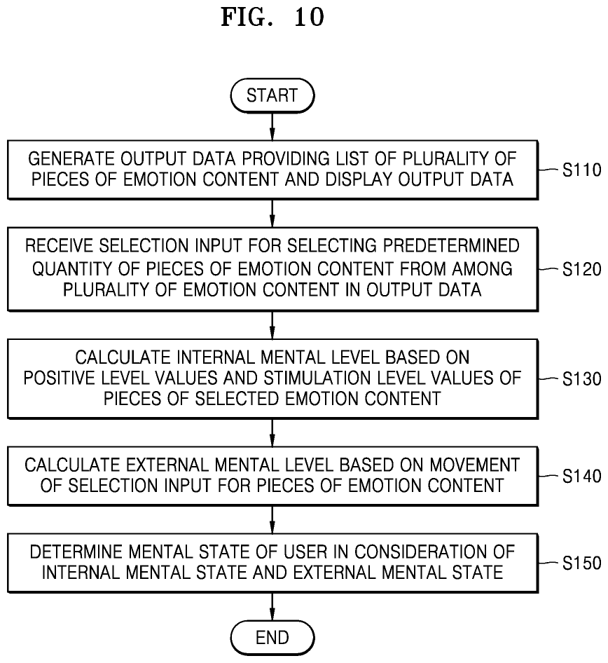

FIG. 10 is a flowchart of a method of determining a mental state of a user in consideration of an external mental level according to an input behavior of the user, according to embodiments of the present disclosure.

In operation S110, the electronic device 100 may generate output data providing a list of a plurality of pieces of emotion content, and display the output data on an outputter.

In operation S120, the electronic device 100 may receive a selection input for selecting a predetermined quantity of pieces of emotion content from among the plurality of pieces of emotion content in the output data.

The electronic device 100 may input a selection input for selecting emotion content from among the output data. The electronic device 100 may measure the selection input and a degree of movement corresponding to the selection input. The degree of movement may be measured based on, when first to fifth emotion content is selected, the number of times that a row moves, the number of times that a row moves, etc. while the first to fifth emotion content is selected.

The electronic device 100 may measure the selection input and a selection time interval of the selection input. The selection time interval of the selection input may be a time interval from the time when the emotion content is initially selected to the time when the selection of the emotion content is finished.

The electronic device 100 may obtain the selection input for selecting the emotion content, the degree of movement, and the selection time interval.

In operation S130, the electronic device 100 ay calculate an internal mental level based on positive level values and stimulation level values of the pieces of selected emotion content.

In operation S140, the electronic device 100 may calculate an external mental level based on a movement of the selection input for selecting the pieces of emotion content.

The electronic device 100 may determine the external mental level based on the selection input for selecting the pieces of emotion content. The external mental level may be determined based on at least one of the selection input, the degree of movement of the selection input, and the selection time interval of the selection input. In another embodiment, the electronic device 100 may calculate a movement speed based on the degree of movement and the selection time interval, and may determine the external mental level based on the movement speed.

In operation S150, the electronic device 100 may determine a mental state of a user in consideration of the internal mental level and the external mental level.

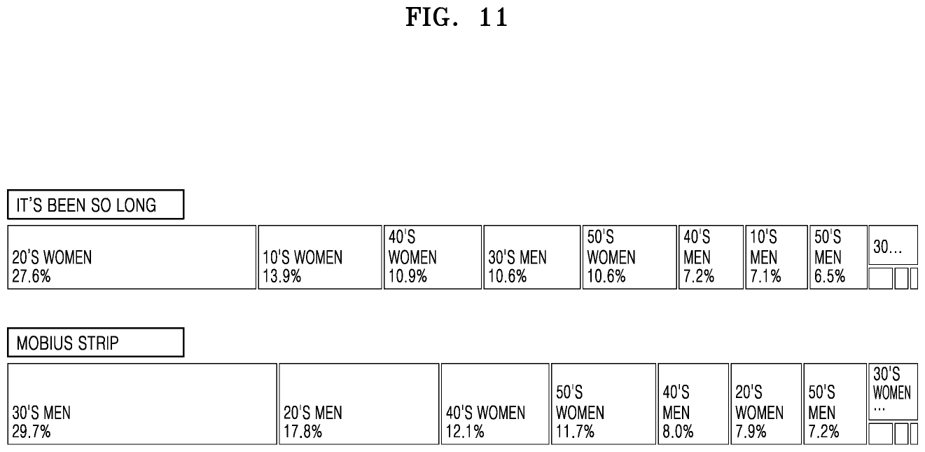

FIGS. 11 to 14 are diagrams of examples of statistical data on pieces of provided emotion content, internal mental levels, and external mental levels, according to embodiments of the present disclosure.

FIG. 11 is an exemplary diagram of statistical data for ‘in a short time’ and ‘Moebius strip’, which are exemplary emotional content.

As illustrated in FIG. 11, a management server (not illustrated) electrically connected or connected via a network to the mentality determiner 110 may collect data obtained through the mentality determiner 110 provided in an electronic device, and generate statistical data for pieces of emotion content based on the collected data. An age and gender ratio of users who selected ‘it's been so long’ may be generated as statistical data. In addition, an age and gender ratio of users who selected ‘Mobius strip’ may be generated as statistical data.

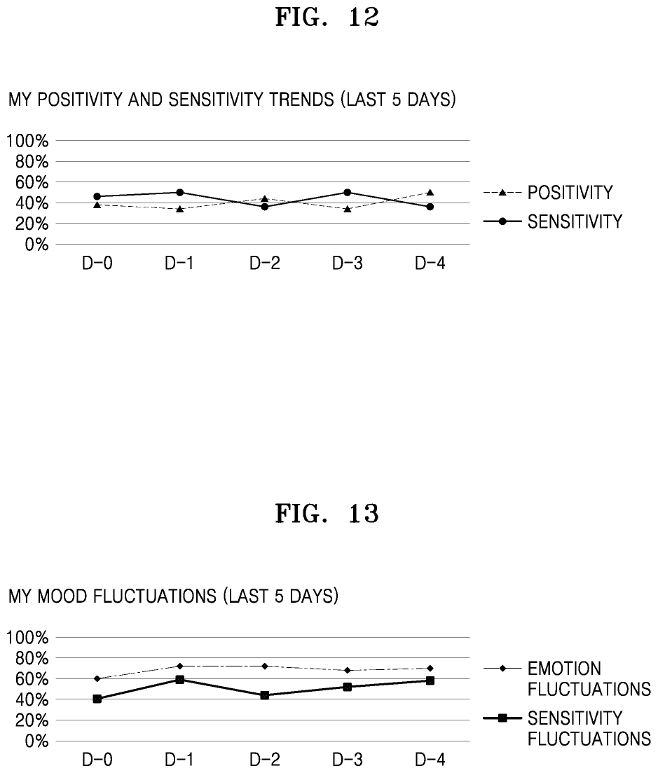

FIG. 12 is an exemplary diagram of a change trend in positivity and sensitivity of a user. As illustrated in FIG. 12, the data processor 115 of the mentality determiner 110 may generate statistical data on pieces of emotion content, an internal mental level, and an external mental level of a corresponding user. The data processor 115 may generate change trends with respect to positive index values and stimulation index values of pieces of selected emotion content. Positivity of the corresponding user may be calculated in response to the positive index values of the pieces of selected emotion content. Sensitivity of the corresponding user may be calculated in response to the stimulation index values of the pieces of selected emotion content.

FIG. 13 is an exemplary diagram of a user's emotional fluctuations. As illustrated in FIG. 13, the data processor 115 may generate change trends of mood fluctuations and sensitivity fluctuations.

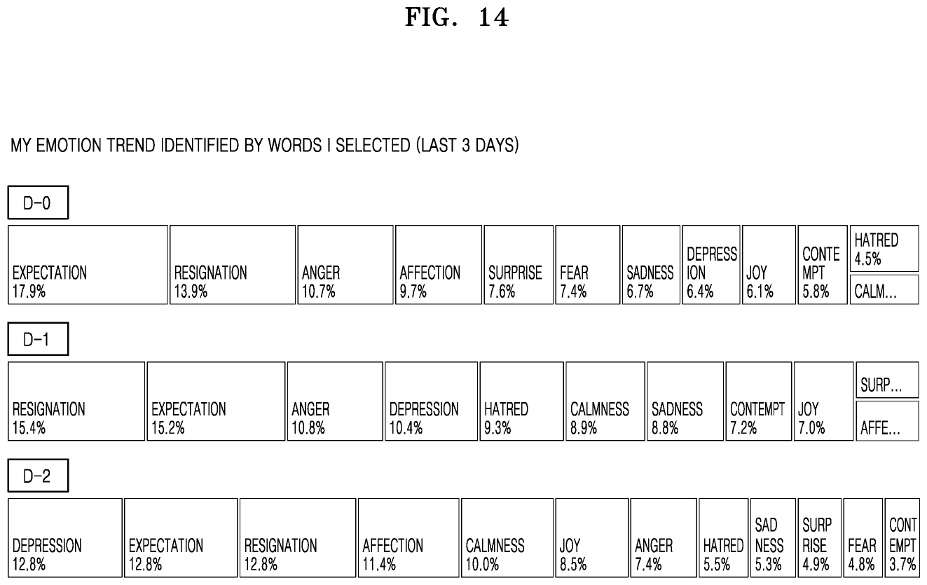

FIG. 14 is an exemplary diagram of a change trend of emotional contents selected by a user. As illustrated in FIG. 14, the data processor 115 may generate ratio information for a first internal mental level as expectation, resignation, anger, affection, surprise, fear, sadness, depression, joy, contempt, hatred, etc. based on emotion coordinate values of pieces of emotion content selected on a first day. The data processor 115 may generate ratio information for a second internal mental level as resignation, expectation, anger, depression, hatred, calmness, sadness, contempt, joy, surprise, etc. based on emotion coordinate values of pieces of emotion content selected on a second day.

The data processor 115 may generate ratio information for a third internal mental level as depression, expectation, resignation, affection, calmness, joy, anger, hatred, sadness, surprise, fear, contempt, etc. based on emotion coordinate values of pieces of emotion content selected on a third day.

As described above, the data processor 115 may generate history information and statistical information for selection inputs performed by users. At the internal mental level, a change trend in depression, a change trend in hatred, a change trend in fear, and a change trend in contempt may be individually generated.

The apparatus described above may be implemented as a hardware component, a software component, and/or a combination of the hardware component and the software component. For example, the apparatus and components described in the aforementioned embodiments may be implemented by using one or more general-purpose or special-purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. A processing device may execute an operating system (OS) and one or more software applications running on the operating system. The processing device may also access, store, manipulate, process, and generate data in response to execution of software. For convenience of understanding, it has sometimes been described that one processing device is used, but one of ordinary skill in the art will recognize that the processing device may include a plurality of processing elements and/or a plurality of types of processing elements. For example, the processing device may include a plurality of processors or one processor and one controller. Other processing configurations are also possible, such as parallel processors.

The software may include a computer program, code, instructions, or a combination of one or more of these, and may configure a processing device or independently or collectively instruct the processing device to perform desired operations. The software and/or data may be permanently or temporarily embodied in any kind of machine, component, physical device, virtual equipment, computer storage medium or device, or transmitted signal wave, to be interpreted by or to provide instructions or data to the processing device. The software may be distributed over networked computer systems and stored or executed in a distributed manner. The software and data may be stored in one or more computer-readable recording media.

The method according to the embodiments may be embodied as program instructions executable by various computer means, and may be recorded in a computer-readable recording medium. The computer-readable recording medium may include, alone or in combination with, a program instruction, a data file, a data structure, etc. The program instructions written to the computer-readable recording medium may be specifically designed and configured for the embodiments or may be well-known and available to one of ordinary skill in the art. Examples of the computer-readable recording medium include magnetic media (e.g., hard disks, floppy disks, magnetic tapes, etc.), optical media (e.g., CD-ROMs, or DVDs), magneto-optical media (e.g., floptical disks), and hardware devices specifically configured to store and execute program instructions (e.g., ROM, RAM, flash memories, etc.). Examples of the program instructions include advanced language codes executable by a computer by using an interpreter or the like as well as machine language codes made by a compiler. The hardware devices described above may be configured to operate as one or more software modules to perform the operations of the embodiments, and vice versa.

According to the embodiments of the present disclosure, the internal mental level of the user may be determined based on stimulation levels and positive levels of one or more pieces of emotion words selected by the input behavior of the user, the external mental level of the user may be determined based on the degree of movement by the input behavior of the user or the movement path, and the mental state of the user may be determined in consideration of the internal mental level and the external mental level.

As described above, although the embodiments have been described with reference to the limited embodiments and drawings, various modifications and variations may be made from the above description by those of ordinary skill in the art. For example, the aforementioned method may be performed in a different order, and/or the aforementioned components, such as systems, structures, apparatuses, circuits, etc., may be combined in different combinations from what is described above, and/or replaced or substituted by other components or equivalents thereof, to obtain appropriate results.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of determining a mental state of a user in consideration of an external mental level according to an input behavior of the user, the method comprising:
generating, by an electronic device, output data providing a list of a plurality of pieces of emotion content and displaying the output data;
receiving, by the electronic device, a selection input for selecting a predetermined quantity of pieces of emotion content from among the plurality of pieces of emotion content in the output data;
calculating, by the electronic device, an internal mental level based on positive level values and stimulation level values of the selected predetermined quantity of pieces of emotion content;
calculating, by the electronic device, an external mental level based on a movement of the selection input for selecting the predetermined quantity of pieces of emotion content; and
determining, by the electronic device, a mental state of the user in consideration of the internal mental level and the external mental level.

2. The method of claim 1, wherein
the output data includes a user interface in which a plurality of pieces of emotion content are arranged in a plurality of rows and a plurality of columns in a predetermined arrangement, and
the arrangement of the plurality of pieces of emotion content is determined by an administrator and is fixed.

3. The method of claim 1, wherein the calculating of the external mental level includes calculating, based on coordinate values of the selected predetermined quantity of pieces of emotion content, a physical distance value between the predetermined quantity of pieces of emotion content and a movement speed value, and calculating the external mental level in consideration of the physical distance value between the predetermined quantity of pieces of emotion content or the movement speed value.

4. The method of claim 1, further comprising calculating, by the electronic device, a degree of relevance between internal and external mental states in consideration of the internal mental level and the external mental level, and calculating a reliability of the internal mental level of the user based on the degree of relevance between the internal and external mental states.

5. The method of claim 4, further comprising changing, by the electronic device, the mental state of the user based on a degree of relevance between internal and external mental states for each user.

6. The method of claim 4, further comprising
calculating, by the electronic device, a degree of relevance between internal and external mental states based on the internal mental level and the external mental level,
when the degree of relevance between the internal and external mental states exceeds a preset first reference value, setting, to ‘high’, the reliability of the internal mental level based on the predetermined quantity of pieces of emotion content selected by the user, and
when the degree of relevance between the internal and external mental states is less than or equal to a second reference value, setting, to ‘low’, the reliability of the internal mental level based on the predetermined quantity of pieces of emotion content selected by the user.

7. The method of claim 6, wherein the first and second reference values are set for each user or set based on at least one of age, gender, occupation, and residential area of the user.

8. The method of claim 1, wherein, from among the predetermined quantity of pieces of emotion content, pieces of emotion content provided at once include pieces of emotion content classified into a predetermined positive level value and stimulation level value.

9. An electronic device comprising:
a provision data generator configured to generate output data providing a list of a plurality of pieces of emotion content and display the output data;
an emotion path inputter configured to receive a selection input for selecting a predetermined quantity of pieces of emotion content from among the plurality of pieces of emotion content in the output data;
an internal mental level determiner configured to calculate an internal mental level based on positive level values and stimulation level values of the selected predetermined quantity of pieces of emotion content;
an external mental level determiner configured to calculate an external mental level based on a movement of the selection input for selecting the predetermined quantity of pieces of emotion content; and
a data processor configured to determine a mental state of a user in consideration of the internal mental level and the external mental level.

10. A computer-readable non-volatile recording medium having a program recorded thereon, the program being executed by a processor to:
generate output data providing a list of a plurality of pieces of emotion content and displaying the output data;
receive a selection input for selecting a predetermined quantity of pieces of emotion content from among the plurality of pieces of emotion content in the output data;
calculate an internal mental level based on positive level values and stimulation level values of the selected predetermined quantity of pieces of emotion content;
calculate an external mental level based on a movement of the selection input for selecting the predetermined quantity of pieces of emotion content; and
determine a mental state of a user in consideration of the internal mental level and the external mental level.
